(19) United States
(12) Patent Application Publication
(10) Pub. No.: US 2008/0045863 A1
(43) Pub. Date: Feb. 21, 2008

(54) Guidewire structure including a medical guidewire

(75) Inventor: Gregory J. Bakos, Mason, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc.

(21) Appl. No.: 11/505,607

(22) Filed: Aug. 17, 2006

(51) Int. Cl. A61M 25/00

(52) U.S. Cl. 600/585

(57) ABSTRACT

A guidewire structure includes a medical guidewire and an attached guidewire anchor having collapsed and expanded states. The anchor includes struts and defines in the expanded state an open structure. Another guidewire structure includes a medical guidewire, an attached guidewire anchor having collapsed and expanded states, and an overtube. The anchor is resiliently flexible and is resiliently biased to the expanded state. The overtube is adapted to slide over the medical guidewire, to slidably cover and compress the anchor, and to slidably expose the anchor. Another guidewire structure includes a medical guidewire, a guidewire anchor, and a hollow tube having a distal end attached proximate the proximal end of the guidewire anchor. The medical guidewire is positioned within the hollow tube and within the guidewire anchor and is attached proximate the distal end of the anchor. The anchor is flexible and has collapsed and expanded states.

### FIELD OF THE INVENTION

The present invention is related generally to guidewire structures, and more particularly to a guidewire structure having a medical guidewire.

### BACKGROUND OF THE INVENTION

A physician typically accesses and visualizes tissue within a patient's gastrointestinal (GI) tract with an endoscope (such as a gastroscope or a colonoscope) having a long, flexible insertion tube. For the upper GI, a physician may insert a gastroscope into the sedated patient's mouth to examine and treat tissue in the esophagus, stomach, and proximal duodenum. For the lower GI a physician may insert a colonoscope through the sedated patient's anus to examine the rectum and colon. Some endoscopes have a working channel in the insertion tube, typically about 2.5-3.5 millimeters in diameter, extending from a port in the handpiece to the distal portion of the insertion tube. A physician may insert medical devices into the working channel to help diagnose or treat tissue within the patient.

Guidewires anchorable by inflatable balloons have been used to aid the introduction of catheters (such as insertion tubes of endoscopes) and other instruments into many sites in the human body. Many medical applications and specific designs of guidewires have been for cardiovascular use. There are, however, specific challenges relating to the use of guidewires in the GI tract, as opposed to the vascular system. Thus, the bowel is more tortuous, softer and generally of larger diameter. Furthermore, in the case of the small intestine and the colon, these are longer than most arteries or veins.

Still, scientists and engineers continue to seek improved guidewire structures having a medical guidewire.

### SUMMARY

A first expression of a first embodiment of a guidewire structure of the invention includes a medical guidewire and a guidewire anchor attached to the medical guidewire. The guidewire anchor has a collapsed state and an expanded state. The guidewire anchor includes a plurality of struts and defines in the expanded state an open structure.

A second expression of a first embodiment of a guidewire structure of the invention includes a medical guidewire, a guidewire anchor attached to the medical guidewire, and an overtube. The guidewire anchor has a collapsed state and an expanded state. The guidewire anchor is resiliently flexible and is resiliently biased to the expanded state. The overtube is adapted to slide over the medical guidewire, to slidably cover and compress the guidewire anchor to the collapsed state, and to slidably expose the guidewire anchor allowing the guidewire anchor to resiliently assume the expanded state.

A first expression of a second embodiment of a guidewire structure of the invention includes a medical guidewire, a guidewire anchor, and a hollow tube. The guidewire anchor has a distal end and a proximal end. The hollow tube has a distal end attached proximate the proximal end of the guidewire anchor. The medical guidewire is positioned within the hollow tube and within the guidewire anchor and is attached proximate the distal end of the guidewire anchor. The guidewire anchor is flexible and has a collapsed state and an expanded state.

Several benefits and advantages are obtained from one or more of the expressions of embodiments of the invention. In a one example of the first expression of the first embodiment, having a flexible guidewire anchor which is attached to a medical guidewire and which includes a plurality of struts and defines in the expanded state an open structure will avoid the leak problems of a balloon guidewire anchor and, especially when the struts include barbs, should provide improved gripping of patient tissue to better anchor the medical guidewire. The same reasoning should apply in an example of the second expression of the first embodiment and in an example of the second embodiment concerning avoiding the leak problems of balloon guidewire anchors and, especially when the guidewire anchor includes barbs, improving gripping of patient tissue.

### BRIEF DESCRIPTION OF THE FIGURES

FIG. 1 is a schematic side-elevational cutaway view of a first embodiment of a medical instrument having a catheter and employing a first embodiment of a guidewire structure of the invention, wherein the guidewire structure has a medical guidewire, has a stent-type, resiliently flexible guidewire anchor resiliently biased to the expanded state, and has an overtube, wherein the medical guidewire is employed as a loop-track guidewire, and wherein the overtube has been pulled to slidingly expose the guidewire anchor allowing the guidewire anchor to resiliently assume the expanded state;

FIG. 2 is a schematic side-elevational view of the distal portion of FIG. 1 but previous in time to FIG. 1 and with the catheter and the overtube shown in cross section, wherein the overtube has been pushed to slidingly cover and collapse the guidewire anchor before the guidewire anchor and a portion of the medical guidewire attached to the proximal end of the guidewire anchor were extended beyond the distal end of the catheter;

FIG. 3 is a schematic side-elevational view of the guidewire structure of FIG. 1;

FIG. 4 is a schematic side-elevational view of the distal portion of a second embodiment of a medical instrument having a catheter and employing an alternate first embodiment of a guidewire structure of the invention, wherein the guidewire structure has a medical guidewire, has a vena-cava-filter-type, resiliently flexible guidewire anchor resiliently biased to the expanded state, and has an overtube, wherein the medical guidewire is employed as a non-loop-track guidewire, and wherein the overtube has been pushed to slidingly cover and collapse the guidewire anchor allowing the guidewire anchor and a portion of the medical guidewire attached to the proximal end of the guidewire anchor to be extended from the distal end of the catheter;

FIG. 5 is a schematic side-elevational view of the guidewire structure of FIG. 4 with the overtube pulled to expose the guidewire anchor allowing the guidewire anchor to resiliently assume the expanded state;

FIG. 6 is a schematic side-elevational view of a second embodiment of a guidewire structure of the invention, wherein the guidewire structure has a stent-type, flexible guidewire anchor, has a hollow tube attached proximate the proximal end of the guidewire anchor, and has a medical guidewire positioned within the hollow tube and within the medical anchor, wherein the guidewire anchor is in a collapsed state; and

FIG. 7 is a view, as in FIG. 6, but with the guidewire anchor in an expanded state.

### DETAILED DESCRIPTION

Before explaining the several embodiments of the present invention in detail, it should be noted that each embodiment is not limited in its application or use to the details of construction and arrangement of parts and steps illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is further understood that any one or more of the following-described embodiments, examples, etc. can be combined with any one or more of the other following-described embodiments, examples, etc.

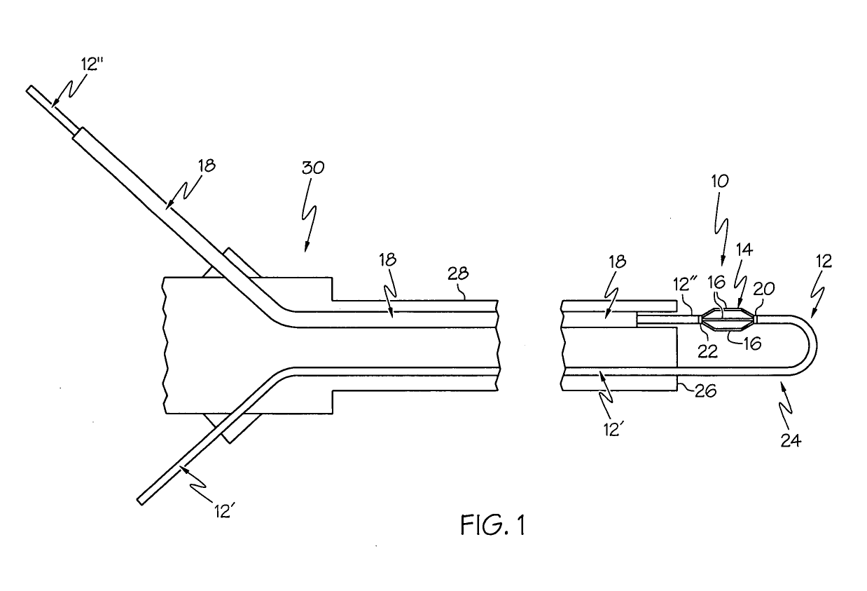

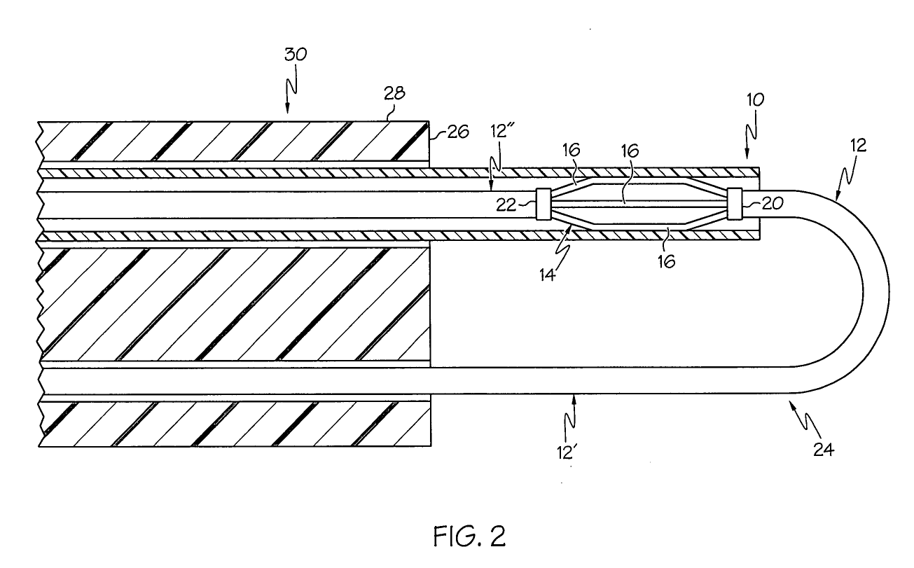

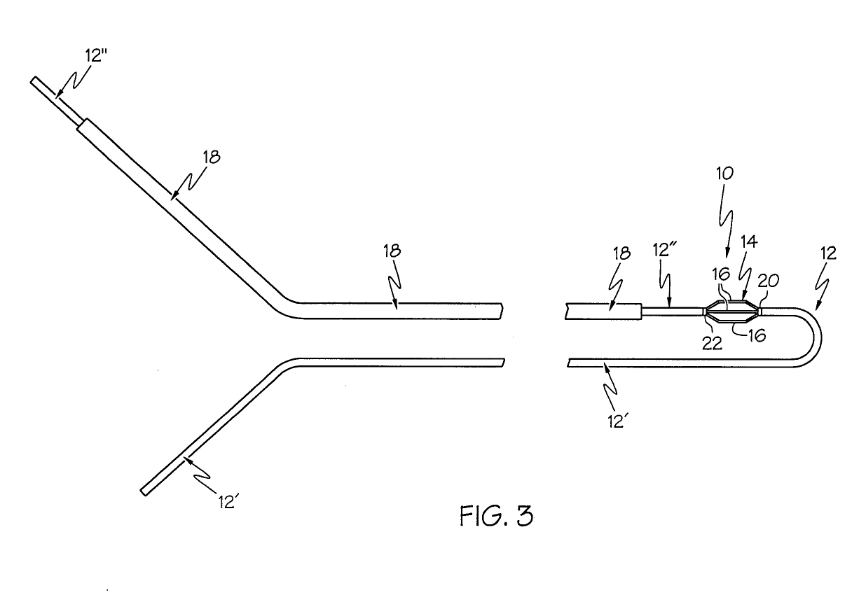

A first embodiment of a guidewire structure 10 of the invention is shown in FIGS. 1-3. A first expression of the guidewire structure 10 of the embodiment of FIGS. 1-3 includes a medical guidewire 12 and a guidewire anchor 14 attached to the medical guidewire 12. The guidewire anchor 14 has a collapsed state (as seen in FIG. 2) and an expanded state (as seen in FIGS. 1 and 3). The guidewire anchor 14 includes a plurality of struts 16 and defines in the expanded state an open structure. It is noted an open structure having struts has a space between at least some of the struts.

In one enablement of the first expression of the embodiment of FIGS. 1-3, the guidewire anchor 14 in the collapsed state and the medical guidewire 12 are insertable into, and slidable within, a body lumen of a patient, and the guidewire anchor 14 in the expanded state is adapted to anchor the medical guidewire 12 in the body lumen. In one variation, the guidewire anchor 14 consists essentially of a shape memory alloy. In one example, the shape memory alloy is nitinol available from Nitinol Devices & Components (Fremont, Calif.). In one method of construction, a nitinol tube is laser cut to create the guidewire anchor 14 including the struts 16.

A second expression of the guidewire structure 10 of the embodiment of FIGS. 1-3 includes a medical guidewire 12, a guidewire anchor 14 attached to the medical guidewire 12, and an overtube 18. The guidewire anchor 14 has a collapsed state and an expanded state. The guidewire anchor 14 is resiliently flexible and is resiliently biased to the expanded state. The overtube 18 is adapted to slide over the medical guidewire 12, to slidably cover and compress the guidewire anchor 14 to the collapsed state, and to slidably expose the guidewire anchor 14 allowing the guidewire anchor 14 to resiliently assume the expanded state.

It is noted that the enablements, variations, and examples, etc. of the first expression of the embodiment of FIGS. 1-3 are equally applicable to the second expression of the embodiment of FIGS. 1-3. In one arrangement, the overtube 18 is a lubricious overtube, such as one consisting essentially of Polytetrafluroethylene (PTFE), such as Striped Teflon® PTFE available from Zeus, Inc (Orangeburg, S.C.).

In one implementation of the second expression of the embodiment of FIGS. 1-3, the guidewire anchor 14 has a distal end 20 and a proximal end 22, wherein the medical guidewire 12 has a first segment 12′ attached to the distal end 20 of the guidewire anchor 14 and has a second segment 12″ attached to the proximal end 22 of the guidewire anchor 14. In one variation, the attachments of the first and second segments 12′ and 12″ to the guidewire anchor 14 are laser welded attachments. In one modification, the medical guidewire 12 is resiliently flexible. In one example, each of the first and second segments 12′ and 12″ is resiliently flexible. In one choice of materials, the first segment 12′ includes a monolithic length of nitinol, and the second segment 12″ includes a monolithic length of nitinol. In one illustration, the monolithic length of nitinol of the first segment 12′ (and optionally the second segment 12″) is covered by an attached (heat-shrunk) lubricious sleeve such as one consisting essentially of Polytetrafluroethylene (PTFE), such as Striped Teflon® PTFE available from Zeus, Inc (Orangeburg, S.C.). In a different implementation, not shown, the medical guidewire lacks the first segment.

In a first arrangement of the second expression of the embodiment of FIGS. 1-3, the guidewire anchor 14 includes a plurality of struts 16 and defines in the expanded state an open structure, and the open structure has a shape of substantially an installed medical stent (see FIGS. 1 and 3). The exact shape of the medical stent is left to those skilled in the art and can include, for example, cross struts and one-way barbs (not shown).

In one application of the second expression of the embodiment of FIGS. 1-3, the medical guidewire 12 is used as a closed-loop guidewire. Here, a working portion 24 of the medical guidewire 12 is extendable beyond a distal end 26 of a catheter 28 of a medical instrument 30 (such as the distal end of a flexible insertion tube of an endoscope such as a gastroscope or a colonoscope), wherein the working portion 24 includes a portion of the first segment 12′ and a portion of the second segment 12″. It is noted that a proximal end portion of the first segment 12′ extends proximally through a first passageway of the catheter 28 and outside the medical instrument 30, and the proximal end portion of the second segment 12″ extends proximally through a second passageway of the catheter 28 and outside the medical instrument 30. In one variation, not shown, the first and second segments extend proximally through a single passageway such as a working channel of an insertion tube of an endoscope. In another variation, not shown, the guidewire structure is disposed completely outside the catheter but is engaged by guide ways on the exterior surface of the catheter. Other variations are left to the artisan.

In one configuration, the length of the guidewire anchor 12 plus the length of the working portion 24 (i.e., a maximum portion of the first segment 12′ plus a maximum portion of the second segment 12″ which can be extended beyond the distal end 26 of the catheter 28) is at least six feet. In one variation, the first and second segments 12′ and 12″ have a substantially circular cross-section having a maximum diameter which is always less than 0.050-inch and a minimum diameter which is always at least 0.010-inch. In one modification, the guidewire anchor 14 is substantially 2.5 inches long, has a maximum span in the expanded state of substantially 1.75 inches, and has a minimum span in the collapsed state of substantially 0.080 inches. In one example, the overtube 18 has an inside diameter of substantially 0.100 inch and an outside diameter of substantially 0.140. Other configurations are left to those skilled in the art.

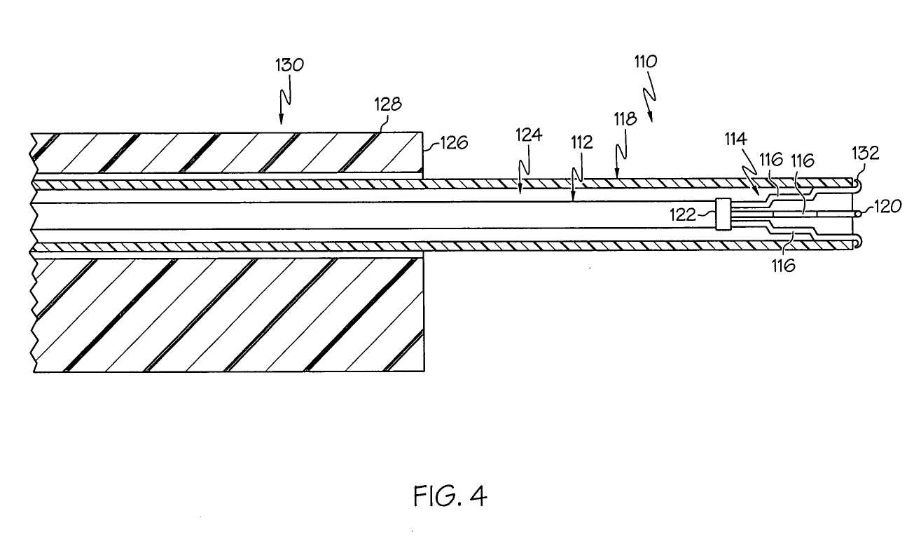

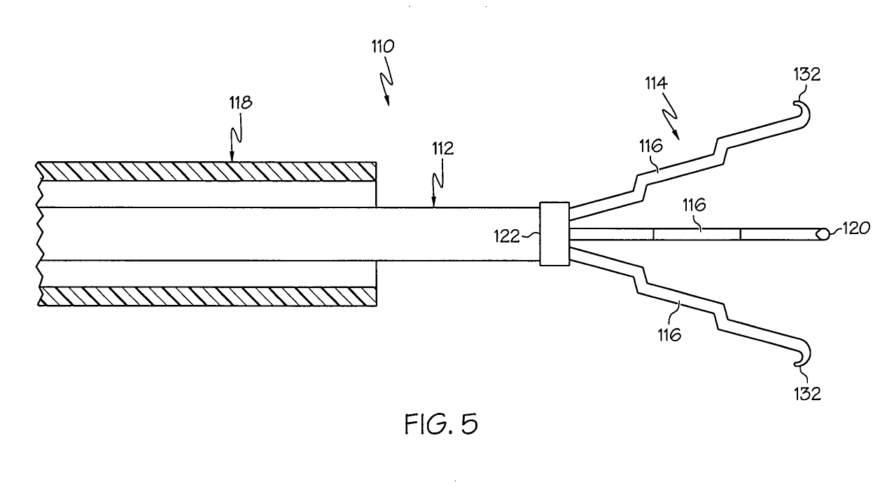

In a second arrangement, as shown in the alternate first embodiment of FIGS. 4-5, the open structure of the guidewire anchor 114 in the expanded state has a shape of substantially an installed medical vena cava filter (see FIG. 5). The exact shape of the medical vena cava filter, such as, for example, the number of struts 116, is left to those skilled in the art. In one variation, the guidewire anchor 114 includes a plurality of one-way barbs 132. The overtube 118 is shown to slidably cover and compress the guidewire anchor 114 to the collapsed state in FIG. 4 allowing the guidewire structure 110 (including a working portion 124 of the medical guidewire 112) to be extended beyond a distal end 126 of a catheter 128 of a medical instrument 130. The overtube 118 is shown to slidably expose the guidewire anchor 114 in FIG. 5 allowing the guidewire anchor 114 to resiliently assume the expanded state. In one illustration, the overtube 118 has an outside diameter small enough to slide within a working channel of a flexible insertion tube of an endoscope.

In FIGS. 4-5, the medical guidewire 112 is used as a non-closed-loop guidewire and is attached proximate the proximal end 122 of the guidewire anchor 114. Closed-loop uses are left to the artisan. In FIGS. 4-5, the distal end 120 of the guidewire anchor 114 (i.e., the distal-most extent of the struts 116) is a free end. It is noted that a guidewire (or other object) attached proximate the proximal end of an anchor is attached closer to the proximal end than the distal end of the anchor. Likewise, a guidewire (or other object) attached proximate the distal end of an anchor is attached closer to the distal end than the proximal end of the anchor.

A method of the invention for using the guidewire structure 10 of FIGS. 1-3 includes steps a) through e). Step a) includes inserting the distal end 26 of the catheter 28 an initial distance into a body lumen of a patient. Step b) includes extending at least a portion of the first segment 12′ beyond the distal end 26 of the catheter 28. Step c) includes extending the guidewire anchor 14 and at least a portion of the second segment 12″ beyond the distal end 26 of the catheter 28 with the overtube 18 covering the collapsed guidewire anchor 14 and the extended second segment 12″. Step d) includes sliding the overtube 14 off the guidewire anchor 14 (and optionally off the extended second segment 12″) exposing the guidewire anchor 14 allowing the guidewire anchor 14 to resiliently assume the expanded state and anchor the medical guidewire 12. Step e) includes advancing the catheter 28 along the extended (and optionally exposed) second segment 12″ further into the body lumen of the patient.

In one implementation of the method, step d) includes manually pulling the overtube 14 slidingly off the covered mechanical anchor 14. In a different implementation, step d) includes using a first motor to pull the overtube slidingly off the covered mechanical anchor 14. In one variation of the first motor implementation, the proximal end of the overtube is always disposed within the catheter and distal a gear box which is driven by a second motor and which engages surface elevational features (such as external threads) on the second segment of the medical guidewire to advance the second segment of the medical guidewire (see United States Patent Application Publication 2005/0256429 for an example of such gearbox and surface elevational features).

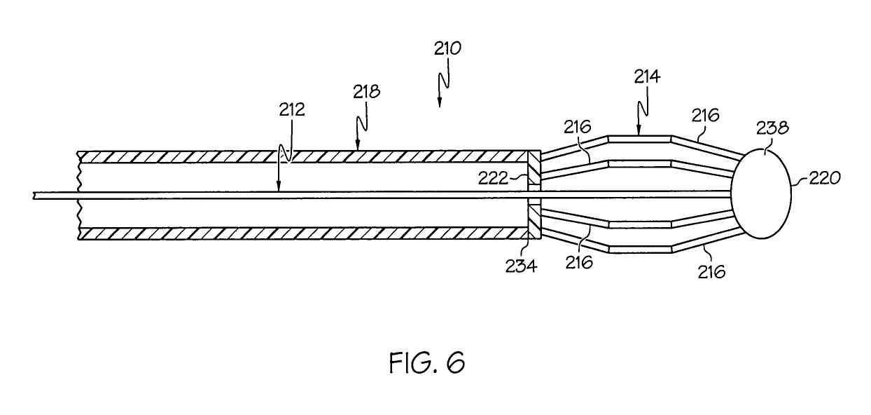

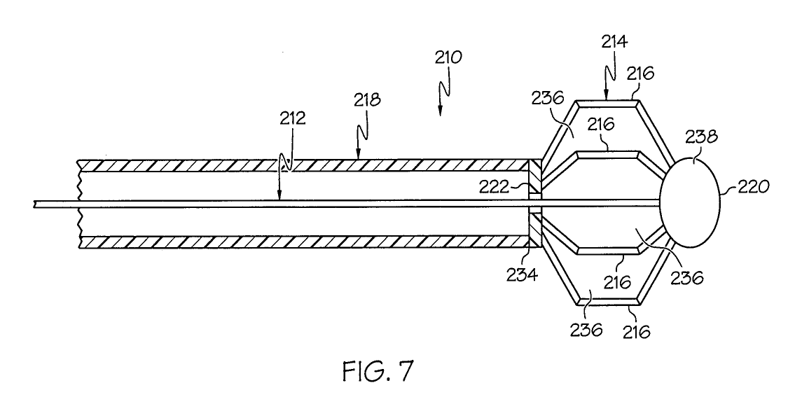

A second embodiment of a guidewire structure 210 of the invention is shown in FIGS. 6-7. A first expression of the guidewire structure 210 of the embodiment of FIGS. 6-7 includes a medical guidewire 212, a guidewire anchor 214, and a hollow tube 218. The guidewire anchor 214 has a distal end 220 and a proximal end 222. The hollow tube 218 has a distal end 234 attached proximate the proximal end 222 of the guidewire anchor 214. The medical guidewire 214 is positioned within the hollow tube 218 and within the guidewire anchor 214 and is attached proximate the distal end 220 of the guidewire anchor 214. The guidewire anchor 214 is flexible and has a collapsed state (as shown in FIG. 6) and an expanded state (as shown in FIG. 7).

In one employment of the first expression of the embodiment of FIGS. 6-7, the guidewire anchor 214 is adapted to collapse (see FIG. 6) when a user pushes on the medical guidewire 212 while pulling on the hollow tube 218. In one variation, the guidewire anchor 214 is adapted to expand (see FIG. 7) when the user pulls on the medical guidewire 212 while pushing on the hollow tube 218. In a different variation, the guidewire anchor 214 is resiliently flexible and is resiliently biased to the expanded state.

In one deployment of the first expression of the embodiment of FIGS. 6-7, the guidewire anchor 214 is adapted to expand (see FIG. 7) when a user pulls on the medical guidewire 212 while pushing on the hollow tube 218. In one variation, the guidewire anchor 214 is adapted to collapse (see FIG. 6) when the user pushes on the medical guidewire 212 while pulling on the hollow tube 218. In a different variation, the guidewire anchor 214 is resiliently flexible and is resiliently biased to the collapsed state.

In one application of the first expression of the embodiment of FIGS. 6-7, the guidewire anchor 214 in the collapsed state and the medical guidewire 212 are insertable into, and slidable within, a body lumen of a patient. In this application, the guidewire anchor 214 in the expanded state is adapted to anchor the medical guidewire 212 in the body lumen. In one variation, the guidewire anchor 214 includes a plurality of struts 216 and defines in the expanded state an open structure. In this variation, by “open structure” is meant that in the expanded guidewire anchor 214, there is a space 236 between at least some of the struts 216. In one modification, the open structure has a shape of substantially an installed medical stent. In the same or a different modification, the guidewire anchor 214 includes an elastomeric distal end portion 238.

Several benefits and advantages are obtained from one or more of the expressions of embodiments of the invention. In a one example of the first expression of the first embodiment, having a flexible guidewire anchor which is attached to a medical guidewire and which includes a plurality of struts and defines in the expanded state an open structure will avoid the leak problems of a balloon guidewire anchor and, especially when the struts include barbs, should provide improved gripping of patient tissue to better anchor the medical guidewire. The same reasoning should apply in an example of the second expression of the first embodiment and in an example of the second embodiment concerning avoiding the leak problems of balloon guidewire anchors and, especially when the guidewire anchor includes barbs, improving gripping of patient tissue.

While the present invention has been illustrated by descriptions of a method, several expressions of embodiments, and examples, etc. thereof, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A guidewire structure comprising a medical guidewire and a guidewire anchor attached to the medical guidewire, wherein the guidewire anchor has a collapsed state and an expanded state, and wherein the guidewire anchor includes a plurality of struts and defines in the expanded state an open structure.

2. The guidewire structure of claim 1, wherein the guidewire anchor in the collapsed state and the medical guidewire are insertable into, and slidable within, a body lumen of a patient, and wherein the guidewire anchor in the expanded state is adapted to anchor the medical guidewire in the body lumen.

3. The guidewire structure of claim 2, wherein the guidewire anchor consists essentially of a shape memory alloy.

4. The guidewire structure of claim 3, wherein the shape memory alloy is nitinol.

5. A guidewire structure comprising a medical guidewire, a guidewire anchor attached to the medical guidewire, and an overtube, wherein the guidewire anchor has a collapsed state and an expanded state, wherein the guidewire anchor is resiliently flexible and is resiliently biased to the expanded state, wherein the overtube is adapted to slide over the medical guidewire, to slidably cover and compress the guidewire anchor to the collapsed state, and to slidably expose the guidewire anchor allowing the guidewire anchor to resiliently assume the expanded state.

6. The guidewire structure of claim 5, wherein the guidewire anchor in the collapsed state and the medical guidewire are insertable into, and slidable within, a body lumen of a patient, and wherein the guidewire anchor in the expanded state is adapted to anchor the medical guidewire in the body lumen.

7. The guidewire structure of claim 6, wherein the guidewire anchor includes a plurality of struts and defines in the expanded state an open structure.

8. The guidewire structure of claim 7, wherein the guidewire anchor consists essentially of a shape memory alloy.

9. The guidewire structure of claim 8, wherein the shape memory alloy is nitinol.

10. The guidewire structure of claim 7, wherein the guidewire anchor has a distal end and a proximal end, wherein the medical guidewire has a first segment attached to the distal end of the guidewire anchor and has a second segment attached to the proximal end of the guidewire anchor.

11. The guidewire structure of claim 7, wherein the open structure has a shape of substantially an installed medical stent.

12. The guidewire structure of claim 7, wherein the open structure has a shape of substantially an installed medical vena cava filter.

13. The guidewire structure of claim 7, wherein the guidewire anchor includes a plurality of one-way barbs.

14. A guidewire structure comprising a medical guidewire, a guidewire anchor, and a hollow tube, wherein the guidewire anchor has a distal end and a proximal end, wherein the hollow tube has a distal end attached proximate the proximal end of the guidewire anchor, wherein the medical guidewire is disposed within the hollow tube and within the guidewire anchor and is attached proximate the distal end of the guidewire anchor, wherein the guidewire anchor is flexible and has a collapsed state and an expanded state.

15. The guidewire structure of claim 14, wherein the guidewire anchor is adapted to collapse when a user pushes on the medical guidewire while pulling on the hollow tube.

16. The guidewire structure of claim 14, wherein the guidewire anchor is adapted to expand when the user pulls on the medical guidewire while pushing on the hollow tube.

17. The guidewire structure of claim 14, wherein the guidewire anchor in the collapsed state and the medical guidewire are insertable into, and slidable within, a body lumen of a patient, and wherein the guidewire anchor in the expanded state is adapted to anchor the medical guidewire in the body lumen.

18. The guidewire structure of claim 17, wherein the guidewire anchor includes a plurality of struts and defines in the expanded state an open structure.

19. The guidewire structure of claim 18, wherein the open structure has a shape of substantially an installed medical stent.

20. The guidewire structure of claim 18, wherein the guidewire anchor includes an elastomeric distal end portion.
